(19) United States
(12) Patent Application Publication
(10) Pub. No.: US 2005/0101961 A1
(43) Pub. Date: May 12, 2005

(54) Bone screws

(76) Inventors: Randall J. Huebner, Beaverton, OR (US); Benone Tarcau, Portland, OR (US)

Correspondence Address: KOLISCH HARTWELL, P.C., 520 S.W. YAMHILL STREET, SUITE 200, PORTLAND, OR, 97204, US

(21) Appl. No.: 10/712,202

(22) Filed: Nov. 12, 2003

(51) Int. Cl. A61F 2/30; A61B 17/56; A61B 17/58

(52) U.S. Cl. 606/72

(57) ABSTRACT

Bone screws, and methods of using bone screws, for compression of a bone. The bone screw may include a head configured to exert an axial force selectively on spaced regions of the bone. The head may be fixed, rotatable, and/or slidable relative to a distal, shank portion of the bone screw.

### FIELD OF THE INVENTION

The invention relates to bone screws. More particularly, the invention relates to bone screws, and methods of using bone screws, for compression of a bone.

### BACKGROUND

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that become damaged should be repaired promptly and properly. Typically, cut or fractured bones are treated using fixation devices, which reinforce the bone and keep it aligned during healing. These fixation devices may include casts for external fixation and/or bone plates and bone screws for internal fixation, among others. For example, one or more bone screws may be placed into a bone to span a discontinuity, such as a fracture of the bone. With this placement, the bone screws may function to hold pieces of the bone together while the bone heals to fuse the pieces across the discontinuity.

Bone screws may be configured to apply a compressive force when placed into a bone. This compressive force may pull or push or otherwise press pieces of the bone together, to promote apposition of broken or cut bone surfaces. Such apposition may assist in fracture reduction, that is, setting the fracture, and/or it may accelerate fusion of the bone pieces, among others. Alternatively, or in addition, the bone screws may compress a bone and a bone-repair device, such as a bone plate.

Compressive forces may be created by differential rates of travel of a bone screw in proximal and distal bone pieces. For example, the bone screw may include a thread with a varying pitch, so that a distal portion of the bone screw advances through bone faster than a proximal portion of the screw, thereby pulling a proximal bone piece toward a distal bone piece. However, a bone screw with a varying pitch may be difficult to place at a predetermined axial position, while exerting a desired amount of axial force. In addition, such a bone screw may exert compressive forces at undesired positions within a bone, which may damage the bone.

The bone screw also may be threaded in a distal portion of the bone screw, and nonthreaded in a proximal portion that includes a head of the bone screw. As the threaded distal portion advances into a distal bone piece, the head may be moved into engagement with a proximal bone piece to exert an axial force on the proximal bone piece, thereby pushing the proximal bone piece toward the distal bone piece and compressing the bone. However, many types of heads may be unsuitable for achieving sufficient purchase on the proximal bone piece without damaging the bone. For example, a head may be configured to apply an axial force to a contact area of the bone that is too small to support the axial force. As a result, the head may tend to break through the contact area, rather than being held in position axially. Alternatively, a head may be configured to distribute its axial force over a larger contact area of the bone but may impart a substantial radial force that splits the bone.

### SUMMARY OF THE INVENTION

The invention provides bone screws, and methods of using bone screws, for compression of a bone. The bone screw may include a head configured to exert an axial force selectively on spaced regions of the bone. The head may be fixed, rotatable, and/or slidable relative to a distal, shank portion of the bone screw.

### BRIEF DESCRIPTION OF THE DRAWINGS

FIG. 1 is a view of a fractured bone, fixed with a plurality of bone screws, with the heads of the bone screws received in the bone, in accordance with aspects of the invention.

FIG. 2 is a partially sectional view of a selected portion of the bone and one of the bone screws of FIG. 1, indicated generally at “2” in FIG. 1.

FIG. 3 is a side elevation view of one of the bone screws of FIG. 1.

FIG. 4 is sectional view of a selected portion of the head of the bone screw of FIG. 3, indicated generally at “4” in FIG. 3.

FIG. 5 is a sectional view of the shank of the bone screw of FIG. 3, taken generally along line 5-5 of FIG. 3.

FIG. 6 is a side elevation view of a second exemplary bone screw having a head configured to be received in a bone, in accordance with aspects of the invention.

FIG. 7 is a side elevation view of a third exemplary bone screw having a head configured to be received in a bone, in accordance with aspects of the invention.

FIG. 8 is a side elevation view of a fourth exemplary bone screw having a head configured to be received in a bone, in accordance with aspects of the invention.

FIG. 9 is a sectional view of a proximal portion of the bone screw of FIG. 8, taken generally along line 9-9 of FIG. 8.

FIG. 10 is a side elevation view of an exemplary drill bit that may be used to drill a hole in bone for placement of one of the bone screws of FIGS. 1-9.

### DETAILED DESCRIPTION

The invention provides bone screws, and methods of using bone screws, for compressing a bone. The bone screw may include a head configured to contact and apply an axial force selectively to spaced or separated regions of the bone. Distributing axial force to spaced regions may improve purchase of the head on the bone and may reduce damage to the bone (e.g., splitting of the bone cause by radial forces). The head may be fixed, rotatable, and/or slidable relative to a distal, shank portion of the bone screw.

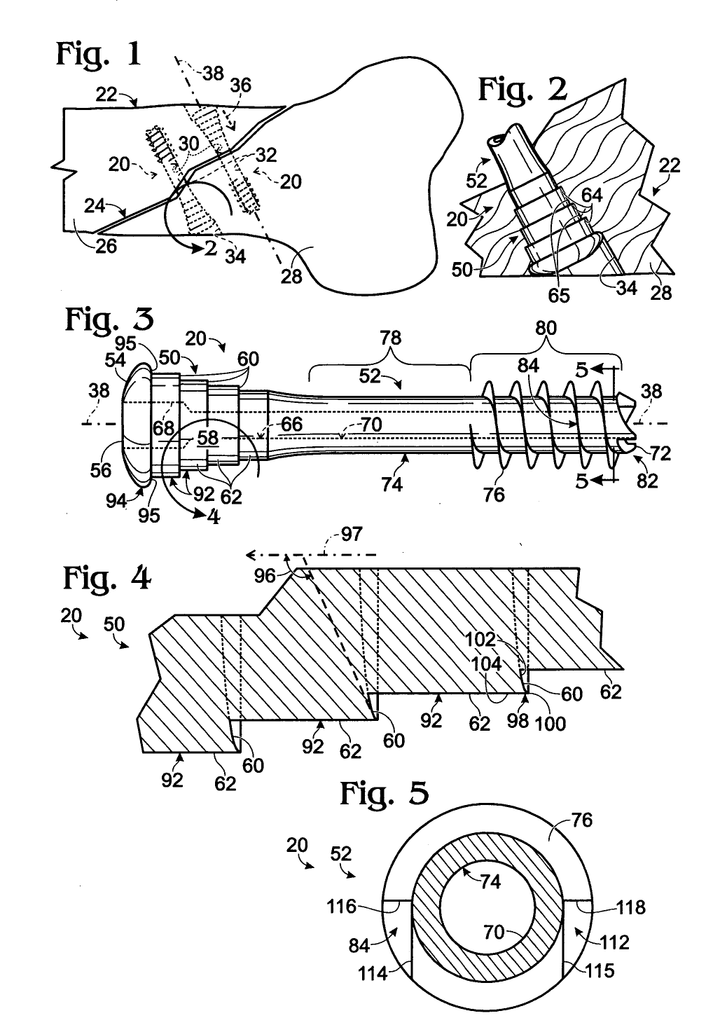

FIG. 1 shows a view of a plurality of bone screws 20, in accordance with aspects of the invention, fixing a fractured bone 22. The bone screws are inside the bone in this view and thus are indicated in phantom outline. Bone 22 may include a fracture 24 or other discontinuity that defines a plurality of bone portions 26, 28. One or more bone screws 20 may be placed into bone 22 to pull bone portions 26, 28 together and fix their relative positions. Bone screws 20 may be disposed in holes 30 of bone 22 so that each screw is positioned at least substantially internal to bone 22. Here, inside or internal to means at least substantially within the natural outside contours of the bone. Each hole 30 may include a smaller bore 32 and a larger counterbore 34 adjoining the smaller bore, to accommodate different parts of the bone screw. The bone screws may apply an axial force 36 (or opposing axial forces) directed parallel to the long or rotation axis 38 of the screws (the screw axis), as the screws are advanced by rotation into bone 22.

FIGS. 2 and 3 show, respectively, a proximal portion of bone screw 20 engaged with bone 22 and a side elevation view of bone screw 20. The bone screw may include a head 50 and a shank 52 joined or coupled, fixedly or movably, to the head.

Head 50 may include surfaces defining the exterior end and exterior sides of the head (see FIG. 3). For example, head 50 may include an axial or end surface 54 defining a proximal end 56 of the bone screw. Head also may include a lateral surface 58 extending distally from end surface 54 toward shank 52 and defining the sides of the head.

Lateral surface 58 may define a plurality of ledge structures or shoulders 60 disposed circumferentially on the head. The ledge structures may be separated by intervening surface regions 62. The ledge structures may be configured to apply an axial force selectively to spaced regions of an apposed bone through contact with the spaced regions. For example, FIG. 2 shows spaced segments 64 of the wall of counterbore 34 in the bone contacting the shoulders of head 50, so that the spaced segments receive a greater axial force than interposed surface regions 65 of the bone.

Head 50 also may define interior surfaces of the bone screw (see FIG. 3). For example, an axial bore 66 may extend along axis 38 to define a tool engagement structure 68. Axial bore 66 also may extend through shank 52, shown at 70, to form a hollow (or cannulated) bone screw.

Shank 52 may define a distal portion of the bone screw including distal end 72. Shank 52 may include a shaft 74 and a thread 76 formed on the shaft, among others. Thread 76 may be disposed on any suitable portion(s) of the shaft. However, in some embodiments, thread 76 may extend along only a portion of the shaft, to define a nonthreaded region or body 78 adjacent the head and a threaded region 80 spaced from the head. Shank 52 may include a tip region 82 that forms a drill, so that the bone screw can be self-drilling in bone, and/or one or more axial flutes 84, so that the bone screw is self-tapping, among others.

Further aspects of the invention are described in the following sections, including (I) overview of bone screws, (II) the head, (III) the shank, and (IV) examples.

I. Overview of Bone Screws

The bone screws described herein may be used on any suitable bone(s), or portions thereof, for any suitable purpose. Suitable bones may include any bone of the human body or of other vertebrate species. Exemplary bones may include bones of the arms (radius, ulna, humerus), legs (femur, tibia, fibula, patella), hands, feet, the vertebrae, the scapula, pelvic bones, cranial bones, and/or the ribs and clavicles, among others. Suitable purposes may include fixation, structural stabilization, attachment of implanted devices, etc. Exemplary uses of the bone screws may include fracture fixation, osteotomy repair, fusion of two or more bones, and/or attachment of bone plates or prostheses to a bone(s), among others.

The bone screws may have any suitable composition and size. The bone screws may be formed of a biocompatible material, such as stainless steel, titanium or a titanium alloy, a cobalt-chromium alloy, a ceramic, a synthetic polymer, a biopolymer, and/or a bioabsorbable material, among others. The bone screws may be sized according to their intended sites of use and their intended functions at those sites. Accordingly, the bone screws may be sized according to a dimension of the bone that will receive a bone screw. For example, smaller screws may be fabricated for use in smaller bones, and larger screws for use in larger bones. In some embodiments, the bone screws may have a length so that the bone screws can be received substantially or completely in a bone. In some embodiments, the bone screws may be sized so that a proximal and/or distal end of the bone screw protrudes somewhat from a bone, for example, to engage a bone plate, a prosthesis, or other implant, among others.

The bone screws may have any suitable external shape. The bone screws may be configured to present an aspect ratio according to an intended use. Generally, however, the bone screws may be elongate, having a length that is substantially greater than their diameter. The bone screws may be based on a circular geometry, so that portions of the bone screws are radially symmetrical. For example, the head and a portion of the shank may be radially symmetric. Alternatively, the bone screws may be radially asymmetric at positions along their length, such as at positions within the shank, where the bone screws are threaded and/or nonthreaded, and/or at positions along the head where ledge or other structures (see below) extend incompletely along a circumferential path around the head.

The bone screws may have any suitable internal shape or surface structure. For example, an internal or interior structure of the bone screws may be defined by an opening, such as a through-hole or a cavity. A through-hole (generally, an axial bore) may have a constant or varying diameter (and/or cross-sectional geometry) along the axis of the screw. In some embodiments, the through-hole may be widened adjacent the proximal end of the screw, to define a counterbore in the head. The counterbore may be used, for example, to provide a larger opening in which a tool, such as a driver, may be received, up to a predefined axial position, to engage the head and rotate the screw. In some embodiments, the head (or a proximal extension of the shank) may include a recess (or other feature), rather than a through-hole, in which a tool such as a driver may be received.

The bone screws may have any suitable indicia. Exemplary indicia that may be suitable include color and/or symbols. Colored indicia may be used to identify a bone screw of a particular size, shape, and/or material, and/or for a particular function, among others. Symbols and/or text also may indicate particular aspects of a bone screw. Other exemplary indicia that may be suitable include reference marks, graduations, surface textures, and/or the like.

Bone screws may be supplied individually and/or in a kit. The kit may include a plurality of bone screws of similar or distinct configurations. Here, distinct screws may be distinguishable based on size, shape, and/or other geometrical characteristics, mobility of the head (e.g., fixed, rotatable, and/or slidable) relative to the shank, etc. The kit also may include a hole-forming tool (such as a drill, a reamer, a drill bit, and/or the like), a driver, bone plates, prostheses, and/or instructions for use of the bone screws, among others.

II. The Head

A bone screw may have a head disposed proximally on the bone screw so that the head follows the distally disposed shank into a bone. The head may be any proximal structure configured to restrict advancement of the bone screw into the bone.

The head may have any suitable size and shape. The head may be enlarged in relation to the shank, that is, configured to have a larger diameter than portions of the shank, particularly a nonthreaded portion of the shank. The head may include this larger diameter along a portion or all of its axial dimension. The diameter of the head may generally decrease from the proximal to the distal end of the head, so that the head narrows distally. Decreases in diameter may be gradual, so that the head tapers, or stepwise. In some embodiments, spaced increases in diameter may be superimposed on a proximal to distal decrease in diameter, for example, to define ridges or other ledge structures, as described further below. The head may extend along any suitable portion of the length of the bone screw, but generally less than half of the length. The length of the head, measured axially, divided by the maximum diameter of the head defines an aspect ratio of the head. Although any suitable aspect ratio may be used, in some embodiments, the aspect ratio of the head may be at least about 1:2 or 1:1. Suitable shapes for the head may deviate somewhat from an ideal mathematical volume, for example, due to the ledge structures. However, exemplary general shapes described by the head may include frustoconical, frustoparaboliodal, frustohyperboloidal, frustospherical, cylindrical, and/or a combination thereof, among others.

The head of the bone screw may have any suitable connection to the shank. The head may be joined to the shank so that the head is fixed in relation to the shank. For example, the head and the shank may be formed unitarily, as a single piece. Alternatively, the head (or a portion thereof) is movable in relation to the shank (or a portion thereof). For example, the head and the shank may be formed as separate pieces (which themselves may have one or more pieces) that may be coupled rotatably to one another.

The head of the bone screw may define a tool engagement structure through which a tool may rotate and advance the bone screw axially. The tool engagement structure may be a hole, such as a socket, or may be a convex surface, and may be configured to be received by or to receive a portion of the tool. The socket or convex surface may be polygonal (such as hexagonal), linear, cruciform, or the like.

The head of the bone screw may include at least one, two, three, or more ledge structures defined by the lateral surface of the head. A ledge structure is any local surface variation of the head configured to engage a bone and provide an increased resistance to axial movement of the head into the bone. The ledge structures may apply a greater axial force than intervening surface regions of the head that separate the ledge structures. Accordingly, a ledge structure may include a ledge surface disposed at a greater angle from the screw axis than an adjacent surface region of the head. For example, the adjacent region may extend parallel to the screw axis or at a first acute angle from the screw axis. The ledge surface may extend at any acute angle when the adjacent surface is parallel or at a second acute angle that is greater than the first acute angle. Alternatively, or in addition, the ledge structure may extend at least substantially perpendicular to the screw axis or at an angle greater than ninety degrees. The angle of a surface, as used herein, is defined as the angular disposition of the surface relative to a distal to proximal axial vector (see FIG. 4).

Ledge structures may extend linearly or nonlinearly from the screw axis. For example, the ledge structure may extend along an arcuate or angular path, so that the surface of the ledge structure is concave or convex along a path from the screw axis.

A ledge structure may extend circumferentially on the head. A structure extending circumferentially on the head, as used herein, means the structure follows a circumferential or closed path on the head. The ledge structure may follow an entire circumferential path, so that the ledge structure extends continuously about the head. Alternatively, the ledge structure may follow a portion of the circumferential path, so that the ledge structure includes one or more gaps and extends discontinuously about the head. The circumferential path may be circular. Accordingly, the ledge structure may describe a complete circle or one or more arcs of a circle. Alternatively, the circumferential path may be partially or wholly noncircular, for example, elliptical, polygonal, wavy, and/or wiggly, among others. However, the path typically will be at least substantially circular, to facilitate turning the screw into the bone.

The ledge structure may be defined by any suitable topography. For example, the ledge structure may be defined by an end of a cylindrical or frustoconical segment, a leading surface of a ridge or other projection, a trailing surface of a groove or other depression, and/or the like.

III. The Shank

A bone screw may have a shank disposed distally on the bone screw so that the shank leads the head into a bone. The shank may be any distal structure configured to promote advancement of the bone screw into a bone.

The shank may have any suitable size and shape. The shank may be elongate. The shank may include, be included in, or be coextensive with, a shaft. The shaft may be solid or hollow. The shaft may be substantially cylindrical or noncylindrical. For example, the shaft may have a constant diameter along its length or may taper distally, among others. Alternatively, or in addition, the shaft may have a circular or noncircular cross section, and the cross section may vary in geometry along the length of the shaft.

The shaft may have any suitable connection to the head. For example, the shaft may be joined to the head, so that the shaft is coextensive with the shank and terminates at the distal end of the head. Alternatively, the shaft may extend partially into the head or through the head, to promote joining separate head and shaft components, and/or to couple the head to the shank rotatably and/or slidably. In some embodiments, the shaft may define a tool engagement structure through which a driver may rotate the shank of the bone screw. The tool engagement structure may include a hole and/or a convex surface, among others, for example, as described above in Section II.

The shank may be threaded. The threaded shank may include a single thread or a plurality of separate threads following generally helical paths. The thread(s) may be continuous or discontinuous, for example, including one or more gaps. The separate threads may be disposed on separate regions of the shank or may be interspersed within the same region of the shank to create a multi-threaded shank. The multi-threaded shank may be configured to have threads with a steeper thread pitch so that the bone screw advances farther with each rotation. The thread(s) may have a constant or varying pitch, and/or a constant or varying diameter, as appropriate. In some embodiments, the thread(s) may define a missing region, or an array of missing regions at registered axial positions of the thread, to create a cutting edge(s) on the thread, so that the bone screw is self-tapping. The cutting edge(s) may be configured to be self-tapping during placement of the bone screw into bone and/or removal of the bone screw out of bone. Alternatively, or in addition, the thread(s) or a subset thereof, may be a machine thread(s) configured to be complementary to a thread(s) in another structure, such as a bone plate or a prosthesis. The thread(s) may be disposed along the entire length of the shank or any suitable portion thereof. In some embodiments, the thread(s) may be restricted to a distal (or proximal) region of the shank so that a proximal (or distal) region is nonthreaded. The threads may have any suitable diameter. In some examples, the diameter of the threads may exceed the diameter of the shaft or shank in the proximal region of the shank but may be smaller than at greatest diameter of the head.

### IV. EXAMPLES

The following examples describe selected aspects and embodiments of the invention, including exemplary bone screws, hole-forming tools for use with bone screws, and methods of placing bone screws into bone. These examples and the various features and aspects thereof are included for illustration and are not intended to define or limit the entire scope of the invention.

### Example 1

### Bone Screw with a Stepped Head

This example describes an exemplary bone screw 20 having a stepped head 50; see FIGS. 1-5. The bone screw includes a stepped head 50 and a shank 52 joined, in this embodiment, fixedly to one another. Aspects of this screw were discussed above, at the beginning of the Detailed Description, particularly in the context of FIGS. 1-3.

FIG. 4 shows a partial sectional view of a portion of stepped head 50 (see FIGS. 1-3 for a full view). Stepped head 50 may be defined by a plurality of cylindrical head segments 92 and an optional cylindrical or noncylindrical most proximal segment 94 that is greater in diameter than head segments 92. Some or all of the head segments may decrease in diameter stepwise toward the shank.

Head segments 92, 94 may define ledge structures 60, 95, respectively. For example, head segments 92 each may define a ledge structure 60 at their distal or leading edge and a cylindrical surface region 62 proximal to or trailing the ledge structure. The ledge structures may be arrayed to generally define a cone. Alternatively, the ledge structures may have an axial spacing and diameters that define a nonconical shape.

FIG. 4 also shows the surface of ledge structure 60 extending in this embodiment at an angle 96 of greater than ninety degrees in relation to a distal-to-proximal vector 97 of the screw axis. Accordingly, ledge structure 60 and adjacent surface region 62 may define a tooth 98 having a rim 100 disposed more distally towards the shank than ledge surface 102 and flanking surface 104. Tooth 98 may be rounded or sharp to define, for example, the ability of the tooth to bite into a bone as the head is advanced against the bone. In sectional profile, ledge surface 102 may be concave, as shown here, or it may be linear or convex, among others.

FIG. 5 shows a sectional view of shaft 74 and thread 76 formed on the shaft near the distal end of the shank 52 of bone screw 20. Thread 76 may define flutes 84, 112. Each flute may be defined by an opening 114, 115 in thread 76. The opening may be a single opening or a plurality of openings, for example, arrayed axially at registered positions of the thread. First opening 114 may have a forward cutting edge 116 configured to cut a thread path into bone as the screw is advanced into bone. Second opening 115 may have a reverse cutting edge 118 configured to cut a thread into bone as the screw is removed from the bone. A bone screw configured to cut its own thread in bone, in one or more rotational directions, may be described as self-tapping.

### Example 2

### Bone Screws with Annular Grooves

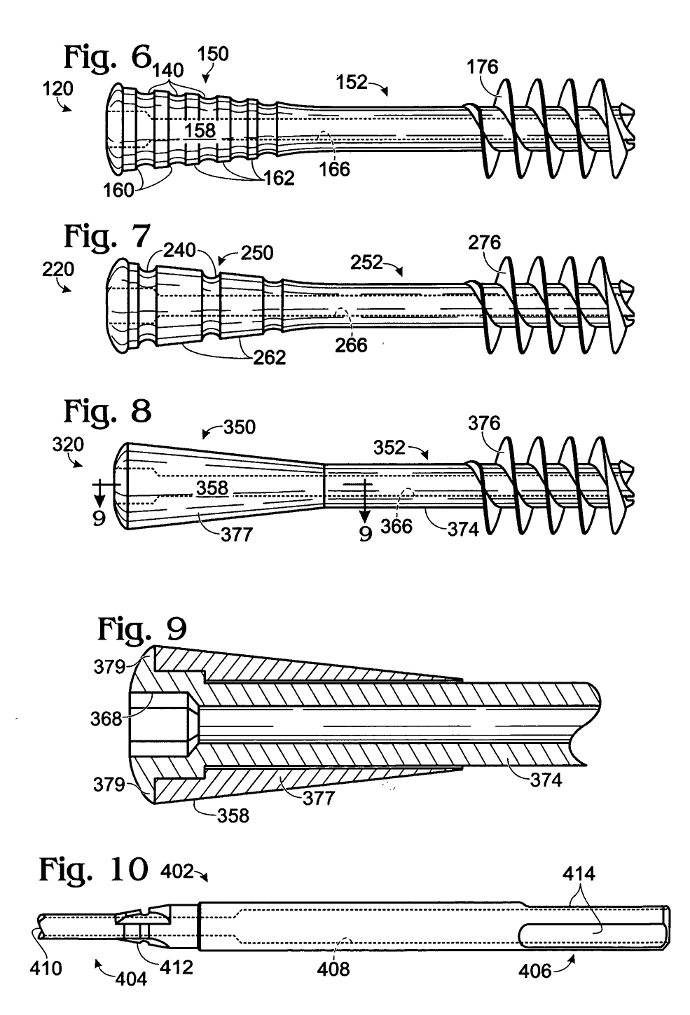

This example describes bone screws 120 and 220 having a plurality of annular grooves disposed on the head; see FIGS. 6-7. For convenience, features of these bone screws have been assigned numerical labels that correspond (in the ones and tens) to those of corresponding features of bone screw 20 in FIGS. 1-5. Thus, for example, heads 150, 250, shanks 152, 252, axial bores 166, 266, and threads 176, 276 of bone screws 120, 220 correspond at least approximately to head 50, shank 52, axial bore 66, and thread 76 of bone screw 20, respectively.

FIG. 6 shows exemplary bone screw 120. Bone screw 120 may include ledge structures 160 created by annular grooves 140 in head 150. Grooves 140 may be defined in lateral surface 158 by depressions in the head flanked by surface regions 162. The ledge structures may be defined by the trailing or proximal sides of each groove 140. In alternative embodiments, the grooves may be configured as ridges that extend from the lateral surface of the head. Furthermore, intervening surface regions may be omitted so that the lateral surface defines an uninterrupted array of ridges and/or grooves.

FIG. 7 shows exemplary bone screw 220. Bone screw 220 may include fewer (or narrower) grooves 240 in head 250 than bone screw 120, described above. Accordingly, intervening surface regions 262 in bone screw 220 may be wider than intervening surface regions 162 in bone screw 120 (see FIG. 6). In alternative embodiments, the bone screws may include more (or wider) grooves in the head than bone screw 120, such that the intervening surface regions are narrower than in bone screw 120.

### Example 3

### Bone Screw with Rotatable Head

This example describes a bone screw having a rotatable head; see FIGS. 8-9. For convenience, features of this bone screw have been assigned numerical labels that correspond (in the ones and tens) to those of corresponding features of bone screw 20 in FIGS. 1-5. Thus, for example, head 350, axial bore 366, tool engagement structure 368, and threads 376 of bone screw 320 correspond at least approximately to head 50, axial bore 66, tool engagement structure 68, and thread 76 of bone screw 20, respectively.

FIGS. 8 and 9 show exemplary bone screw 320. Head 350 of bone screw 320 may be coupled rotatably to shank 352. In particular, in this embodiment, some or all of the lateral surface 358 of the head may rotate in relation to shaft 374. Head 350 may include a sleeve 377 through which shaft 374 extends. Shaft 374 may widen proximally to define a flange 379 that restricts axial movement of the sleeve in a proximal direction (see FIG. 9). Flange 379 may be formed integrally with the shaft or attached as a separate component to the shaft. Sleeve 377 may be configured to slide onto shaft 374 from the distal end of the shaft. Accordingly, the shaft may include no retainer structure to restrict distal movement of the sleeve. Alternatively, the shaft may include a flange or other retainer structure to restrict distal movement of the sleeve.

### Example 4

### Hole-Forming Tool for Bone Screws

This example describes a drill bit 402 that may be used to form a hole for the placement of bone screws, such as those described herein; see FIG. 10.

Drill bit 402 may include a cutting portion 404 and an interface portion 406, among others.

Cutting portion 404 may be configured to cut or form holes adapted to receive bone screws. The cutting portion may include a boring structure 410 to create a hole to receive the shank of the screw. The cutting portion also may include a cutting structure 412 to widen the hole and create a counterbore adjoining the hole. Cutting structure 412 may be configured to create a counterbore having or lacking surfaces complementary to the ledge structures of the screw head. For example, by including or omitting tiers in the cutting surface, cutting structure 412 may be configured to create a stepped or a nonstepped counterbore, among others, to receive head 50 of bone screw 20 (see FIG. 2).

Interface portion 406 may be configured to interface with a person and/or another tool. For example, the interface portion may include a handle so that the drill bit may be operated manually. Alternatively, the interface portion may include surfaces 414 for engagement by a manually or power-driven driver.

Drill bit 402 also may include additional features. For example, the drill bit may include a passage 408 extending through the cutting and interface portions, such that the drill bit is cannulated. Passage 408 may receive a wire placed into a bone, allowing the drill bit to be guided along a predefined path in the bone.

### Example 5

### Method of Bone Screw Placement

This example describes an exemplary method of placing bone screws into a bone. The method may be used to compress a plurality of bone portions (or bones) and/or to pull a bone-repair device (such as a plate, a prosthesis, etc.) and a bone together, among others.

The method may include forming a hole in the bone. The hole may be formed by a bone screw (such as a self-drilling bone screw) and/or by a hole-forming tool, such as a drill. The hole may include a bore to receive the shank of the screw and an optional counterbore to receive the head of the screw.

The method also may include advancing the shank and the head of the bone screw into the hole. The step of advancing may cause the shank to engage a first portion of the bone and/or to be coupled to a bone-repair device. The step of advancing also may cause the head of the bone screw to apply an axial force selectively to a plurality of spaced regions of the bone to which the head is apposed. The spaced regions may be separated by interposed regions of the bone to which a smaller axial force or at least substantially no axial force is applied (for example, using bone screw 20 with cylindrical head segments). The axial force may be applied selectively to a second portion of the bone so that the step of advancing pulls the first and second bone portions toward each other. Alternatively, or in addition, the axial force may pull the bone and the bone-repair device toward one another. The bone screw may be used alone or in combination with other similar or dissimilar bone screws or other fasteners.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A bone screw for compression of a bone, comprising:
a shank including a thread; and
a head connected to the shank and configured to exert an axial force selectively on a plurality of spaced regions of the bone that are apposed to the head.

2. The bone screw of claim 1, the head including a lateral surface defining a plurality of ledge structures and one or more intervening surface regions disposed between the ledge structures, wherein the plurality of ledge structures are configured to apply a greater axial force than the one or more intervening surface regions.

3. The bone screw of claim 2, the shank defining an axial vector extending from the shank toward the head, wherein the plurality of ledge structures and the one or more intervening surface regions extend at an angle from the axial vector, and wherein the angle is greater for the plurality of ledge structures than for the one or more intervening surface regions.

4. The bone screw of claim 3, wherein the angle is at least about 90 degrees for the ledge structures.

5. A bone screw for compression of a bone, comprising:
a shank including a thread; and
a head connected to the shank and including a lateral surface defining a plurality of ledge structures disposed circumferentially on the head.

6. The bone screw of claim 5, wherein the shank has a proximal portion adjacent the head and a distal portion spaced from the head, and wherein the thread is restricted to the distal portion.

7. The bone screw of claim 5, wherein the thread defines an opening so that the bone screw is self-tapping.

8. The bone screw of claim 5, wherein the shank includes a tip region configured to cut a hole in the bone as the bone screw is advanced into the bone.

9. The bone screw of claim 5, wherein the ledge structures are defined by at least one of a plurality of ridges and a plurality of grooves.

10. The bone screw of claim 5, the ledge structures defining spaced circumferential paths, wherein at least one of the ledge structures extends continuously along its circumferential path.

11. The bone screw of claim 10, wherein the at least one ledge structure describes a complete circle.

12. The bone screw of claim 10, wherein the at least one ledge structure describes a portion of a circle.

13. The bone screw of claim 5, wherein the diameter of the plurality of ledge structures decreases successively toward the shank.

14. The bone screw of claim 5, wherein the lateral surface generally describes a frustum of a cone.

15. The bone screw of claim 5, wherein the head includes a plurality of steps defined by stepwise decreases in the diameter of the head.

16. The bone screw of claim 5, wherein at least one of the plurality of ledge structures is included in an annular tooth formed by flanking surface regions of the lateral surface that join at a rim, the rim being disposed closer to the shank than the flanking surface regions.

17. The bone screw of claim 5, wherein the shank and the head define opposing ends of the bone screw and further define an axial bore extending between the opposing ends.

18. The bone screw of claim 17, wherein the axial bore includes a widened region configured to receive a tool that engages the head.

19. The bone screw of claim 5, wherein the head is fixedly connected to the shank.

20. The bone screw of claim 5, wherein the head is rotatably and/or slidably connected to the shank.

21. A bone screw for compression of a bone, comprising:
a shank including a proximal region, a distal region, and a thread restricted to the distal region; and
a head connected to the shank and spaced from the thread by the proximal region, the head including a lateral surface defining a plurality of spaced ledge structures disposed on the head, each ledge structure describing at least portion of a circle.

22. The bone screw of claim 21, wherein the ledge structures are defined by at least one of a plurality of ridges and a plurality of grooves.

23. The bone screw of claim 21, wherein the ledge structures describe complete circles.

24. The bone screw of claim 21, wherein the head includes a plurality of steps defined by stepwise decreases in the diameter of the head.

25. The bone screw of claim 21, wherein the head is generally frustoconical in shape.

26. The bone screw of claim 21, the head having an aspect ratio defined by the axial length of the head relative to the maximum diameter of the head, wherein the aspect ratio is at least 1:1.

27. A bone screw for compression of a bone, comprising:
a shank including a thread; and
a head connected rotatably to the shank and configured to follow the shank into the bone, the head having an aspect ratio defined by its axial length relative to its maximum diameter, the aspect ratio being at least 1:1.

28. A method of compressing a bone with a bone screw, comprising:
forming a hole in the bone;
selecting a bone screw having a shank and a head; and
advancing first the shank and then the head of the bone screw into the hole so that the head contacts and applies an axial force selectively to a plurality of spaced regions of the bone, such that portions of the bone near the head are compressed toward portions of the bone near the shank.

29. The method of claim 28, the spaced regions being separated by interposed regions of the bone, wherein the step of advancing also applies an axial force to the interposed regions, the axial force applied to the interposed regions being less than the axial force applied to the plurality of spaced regions.

30. The method of claim 28, the spaced regions being separated by interposed regions of the bone, wherein the step of advancing also applies no substantial axial force to the interposed regions.

31. The method of claim 28, wherein the step of forming a hole includes forming a bore and a counterbore, and wherein the step of advancing the bone screw disposes the head and the shank at least substantially in the counterbore and the bore, respectively.

32. The method of claim 28, wherein the step of forming a hole is performed by the step of advancing a bone screw.

33. The method of claim 28, wherein the portions of the bone near the head and the portions of the bone near the shank are initially separated by a fracture in the bone.
